(19) United States
(12) Patent Application Publication
(10) Pub. No.: US 2022/0184314 A1
(43) Pub. Date: Jun. 16, 2022

(54) NEEDLELESS INJECTOR AND RELATED METHODS

(71) Applicant: AIJEX PHARMA INTERNATIONAL INC., Toyama City, Toyama Prefecture (JP)

(72) Inventors: Jim M. Fukushima, Orange, CA (US); Tooshikazu Todo, Namerikawa-shi, Toyama (JP)

(73) Assignee: AIJEX PHARMA INTERNATIONAL INC., Toyama City, Toyama Prefecture (JP)

(21) Appl. No.: 17/599,365

(22) PCT Filed: Mar. 27, 2020
(86) PCT No.: PCT/US2020/025449

§ 371 Date: Sep. 28, 2021

Related U.S. Application Data
(60) Provisional application No. 62/826,768, filed on Mar. 29, 2019.

(51) Int. Cl. A61M 5/30; A61M 5/315

(52) U.S. Cl. CPC A61M 5/30; A61M 2207/00; A61M 5/3156; A61M 5/31581

(57) ABSTRACT

A needleless injector having a drive end with an energy source for propelling a quantity of fluid out a distal opening of a barrel of an ampule, wherein the drive end is provided with a cam and a follower for resetting the spring to enable multiple injections and deliveries of a discrete quantities of fluids out the barrel. The drive end can further include a carrier having a stepped barrel for sequentially moving a different surface of a different step adjacent the plunger of the ampule upon each reset of the spring.

### FIELD OF ART

The present disclosure is generally related to a needleless injector with specific discussions on a needleless injector for use with an ampule for delivering medicinal fluids without a needle and wherein the drive end of the injector can incrementally move the plunger of the ampule to deliver multiple dosages to provide multiple-shots, and related methods.

### BACKGROUND

Jet injection devices administer intramuscular and subcutaneous medications without the use of needles. Jet injection devices are also known as needleless injectors. Among the many advantages of jet injection are the reduction of pain and apprehension associated with needles, the elimination of needle stick injuries and the reduction of environmental contamination associated with needle disposal. Jet injection devices are useful in a wide range of drug therapies including immunization vaccines, hormones and local anesthetics, as well as the administration of insulin to the diabetic population, where individuals may require a number of daily injections. Thus, their use has become of increasing interest, particularly by persons of limited physical ability such as the elderly, or the very young.

Injectors, injection drivers, or drive ends for imparting pressure to ampules filled with a fluid medicament may be equipped with a gas cartridge, such as a CO₂ cartridge, or a spring source for driving a piston to generate the needed pressure. In the event an injector is intended for one-time disposable use, the cartridge and/or the spring source makes recycling the injector a difficult task, if not impossible.

### SUMMARY

A needleless injector comprising a drive end comprising a housing, a piston, a compression spring, and a cam and follower assembly.

The cam can comprise a cam surface for moving a follower along a first axis, which can be parallel with a lengthwise axis of the housing.

The needleless injector can further comprise a carrier comprising a stepped barrel and a stem.

The stepped barrel can comprise a plurality of steps and wherein a trigger length between a first step and a second step is approximately equal a trigger length between the second step and a third step.

The piston can displace along a first axis an amount that is substantially equal to the trigger length between the first step and the second step.

An ampule can attach to the housing. The ampule can contain a quantity of medicinal fluid. The ampule can have a barrel and a plunger.

A multi-shot needleless injector comprising a drive end comprising a housing and an ampule, wherein the housing has an interior space and wherein a piston, a compression spring, and a cam and follower assembly are located at least in part inside the housing. Said cam and follower assembly comprising a cam having a slot and said follower comprising a rod, shaft or pin.

A method of performing a needleless injection using the needleless injector of any preceding claim.

A method of performing multiple injections using a needleless injector, said method comprising pushing a trigger to perform a first injection, pushing a cam to reset a spring, and pushing the trigger to perform a second injection.

The first injection and the second injection can be performed with a same ampule, said ampule comprising a barrel and a plunger.

The method can further comprise pushing on the cam to reset the spring and pushing the trigger to perform a third injection with the ampule.

A needleless injector for dispensing a quantity of fluid without a needle, said needleless injector comprising a drive end comprising a housing, a piston, a compression spring, and a cam and follower assembly located inside the piston; and wherein a follower of the cam and follower assembly is arranged to displace along a first direction and a cam of the cam and follower assembly is arranged to displace along a second direction, angled to the first direction.

The cam can comprise a cam surface for moving the follower along the first direction, which is parallel with a lengthwise axis of the housing.

A carrier comprising a stepped barrel and a stem can be in the housing of the drive end and in dynamic arrangement with the piston.

The stepped barrel can comprise a plurality of steps and wherein a trigger length between a first step and a second step can be approximately equal to a trigger length between the second step and a third step.

The piston can be displaceable along a first axis an amount that is substantially equal to the trigger length between the first step and the second step.

An ampule can attach to an opening of the housing of the drive end.

A stem can extend from the stepped barrel. The stem can comprise a gear. The gear on the stem can comprise teeth formed around an elongated body of the stem.

A reset plate can extend out a side of the housing.

One or more springs can bias against the reset plate.

The piston can abut against a rear surface of the stepped barrel.

A further aspect of the invention is a method of performing multiple injections using a needleless injector, said method comprising pushing a trigger to perform a first injection, pushing a cam to reset a spring, and pushing the trigger to perform a second injection.

The method can comprise the step of rotating a carrier comprising a stepped barrel, said stepped barrier can be in contact with a plunger.

The first injection and the second injection can be performed with the same ampule, said ampule can comprise a barrel and a plunger.

The method can comprise the steps of pushing on the cam to reset the spring and pushing the trigger to perform a third injection with the ampule.

A still further aspect of the invention is a method of making a needleless injector, said method comprising assembling an ampule to a housing of a drive end; wherein said ampule comprises a plunger with a plunger tip slidably disposed in an interior of an ampule housing; said housing comprising a piston, a compression spring, and a cam and follower located inside the piston; and wherein a follower of the cam and follower assembly is arranged to displace along a first direction and a cam of the cam and follower assembly is arranged to displace along a second direction, angled to the first direction.

A needleless injector provided in accordance with aspects of the invention can have a drive end and an ampule. The ampule may embody prior art ampules, which can have an ampule housing with an engagement end for attaching to the drive end and a remote end, which has a discharge tip.

The ampule housing can be generally cylindrical with a tiny opening at the discharge tip where a fluid, such as liquid medicine, is expelled to inject the skin of a patient without a needle. The ampule is typically made from a thermoplastic or a composite material.

A plunger with a plunger tip can be slidably disposed inside the ampule housing. When the plunger is pushed or impacted by a piston of the drive end, the piston can accelerate the plunger in the distal direction inside the ampule housing to then dispense a quantity of fluid, such as medicine or other injectable fluids, out the discharge tip of the ampule.

The drive end, which may also be referred to as an injector or an injection driver, can be provided with a pressure or power source for driving a piston against the plunger of the ampule to discharge a quantity of fluid contained inside the ampule. The ampule, which can be like one of the ampules described in WO 2014/042930, PCT/US2013/058013, the contents of which are expressly incorporated herein by reference, can have a barrel and a movable plunger located therein.

The drive end of the present invention can be configured for moving or displacing a piston in increments so that the piston can move against the plunger of the ampule in increments to deliver fluids contained inside the barrel of the ampule in distinct dosages. Said differently, the present drive end is configured to drive a piston in multiple increments to then drive the plunger in increments to discharge fluids inside the ampule in finite quantities or dosages.

In an example, the drive end comprises a housing having a plurality of components contained therein. In an example, the housing is sized and shaped to accommodate components comprising a helical spring, which can be a compression spring, located inside a spring chamber, which can be closed off by a set screw or end cap. In an example, the set crew can be adjusted within a recessed chamber to control the depth of how far the set screw seats within the chamber to then control the amount of compression of the spring, which allows some adjustability of the spring along the spring curve, and therefore the force that the spring exerts against the piston.

In an example, the piston is position through the coil center of the plurality of coils of the spring. The end cap can be adjusted, such as threaded, to move distally or proximally within the recessed chamber. The piston can have a slot or an opening. The slot or opening can be elongated. The piston can have a distal end in abutting contact with a carrier, as further discussed below.

The piston can align to a cam and follower assembly, which has a cam and a follower. The cam can have a structure with a cam surface that resembles a hypotenuse of a right triangle. The follower can be a rod, shaft or pin and can be in dynamic communication with the cam surface. If the housing has a lengthwise axis, the follower can be configured to traverse along the lengthwise axis while the cam is configured to displace radially of the lengthwise axis. For discussion purposes, the lengthwise axis may be called the first axis and movement radial of the lengthwise axis may be called a second axis. This arrangement allows the cam to move along the second axis to cause the follower to move in the first axis, and vice-versa.

Thus, the follower is configured to move a lengthwise distance equal to the two ends of the slot of the cam, which can be called or labelled a shot displacement distance. That is, when the drive end is triggered to release the spring, the piston is configured to move a shot displacement distance to move the follower the same shot displacement distance.

In an example, the follower can be secured to the piston. For example, the follower can project through an opening or slot on the piston. Thus, movement of the piston, the follower, and the cam can be tied together or dependent on one another. In an example, there can be two similar cams located on either side of the piston. For example, each cam can embody a generally rectangular plate that extends about the width of the housing. Each plate can have a cut-out having a cam surface. Each plate can displace along the second axis, perpendicular to the lengthwise axis of the housing. Thus, after the piston is triggered and move along the first axis, or lengthwise axis of the housing, the cams are permitted to move along the second axis. Then as the cams are pushed along the second axis following the trigger, actions of the cams are used to cause the follower to move along the first axis to reset the spring to enable a second trigger, and so forth.

In an example, a reset plate can be provided to reset the cams. The reset plate can be secured to the two cams via a plurality of set screws or another fastener means. One or more compression springs can be provided between the reset plate and a shoulder formed inside the housing, which may be considered or called a return spring or return springs, depending if more than one return spring is used. The return springs push the reset plate outwardly to provide the user with a projected or protruded surface for resetting the primary spring. For example, pressing on the reset plate can impart a force along the second or radial axis. This then can cause the follower to move from the left most position inside the cam to a right most position inside the cam or causes the follower to move along the first axis, or along the lengthwise axis. This then can cause the piston to also move from left to right, when viewing FIG. 1, to reset the primary spring.

A pivotable trigger can be provided to maintain the spring in the loaded or cocked position. For example, a trigger tip can be provided to abut the cam, or a structure connected to the cam to retain the cam until the trigger is depressed or triggered.

A carrier provided herein can comprise a stepped barrel and a stem. The carrier can be provided inside the housing and can be used to generate discrete axial displacements of the plunger located inside the ampule. The stepped barrel can have a front surface and a rear surface defining a length therebetween.

Within the barrel or formed as part of the body of the barrel, steps can be provided similar to steps on a winding staircase. The gaps between each step of the multiple steps on the stepped barrel defines a length along the first axis. Movement of the barrel can cause the movement of the plunger having a plunger tip a shot displacement distance, which can be sized to deliver a desired quantity or volume of fluid out the barrel. For example, at the start of an injection cycle when the barrel is filled with a fluid to be injected and the plunger is at its most rearward or proximal position, the plunger can seat against the lowest, or right most, step of the carrier.

After the plunger is advanced a shot displacement distance by the piston as the spring is released by the trigger, the plunger moves deeper into the barrel by approximately the same amount within the barrel to displace a quantity of fluid out the distal end of the barrel.

When the spring is reset by pressing on the reset plate discussed above, the stem is rotated so that the next step is rotated to be next to the proximal end of the plunger. The gap or distance between the two steps can be approximately equal to the shot displacement distance. Then after the plunger is again advanced a shot displacement distance by the piston, the plunger moves further deeper into the barrel by approximately the same amount within the barrel to displace a second quantity of fluid out the distal end of the barrel.

When the drive end is again reset by pressing on the reset plate, the stem is rotated to rotate the stepped barrel to move the next step adjacent the proximal end of the plunger, which has been displaced in the prior step. At this position, the drive end can fire again to deliver a third shot out the distal end of the barrel without a needle. The process can repeat until the barrel is spent or completely discharged of fluids contained therein. The number of shots can be designed into the barrel by providing the number of steps for generating the number of shots. Three shots can be generated with the three steps. However, fewer or more than three steps can be incorporated.

In an example, there can be two or more steps formed with the stepped barrel. For example, there can be four steps for performing four injections or for displacing four distinct shots or quantities of fluids out the distal end of the barrel. Said differently, there can be four steps so that a contact surface of each of the four steps can push against proximal end most of the plunger to deliver four distinct fluid deposits out the distal end of the ampule. In other examples, there can be more than four steps, such as six steps, eight steps, or ten steps.

In an example, the stem comprises a gear comprising a plurality of teeth, or multiple individual tooth. The gear can be configured to cooperate with the cams, or with one or more levers formed with the one or more cams. When the cams are pushed to reset the spring, as discussed above, the one or more levers push against the gear to rotate the stem to then rotate the stepped barrel so that the next increment step is rotated for pushing against the proximal end edge of the plunger. In an example, each reset of the spring can cause the one or more levers on the cam to push against one tooth to rotate the stem. In other words, each tooth can be pushed serially one at a time during each reset of the spring.

While the plunger moves deeper into the barrel of the ampule following each injection, generated by propelling the stepped barrel with the spring when the trigger is pressed, the present needleless injector can be reset to perform multiple injections with the same ampule such that the first injection and the last injection among the two more injections can be performed with the same drive end without having to remove the ampule in between two successive injections. Further, by controlling the shot displacement distance and the spacing between two different steps on the stepped barrel, the multiple shots can each have roughly the same quantity or volume of fluid dispensed out the discharge end of the barrel.

The trigger can be depressed, and the trigger tip can be spaced from the cam, which has been displaced outwardly so that the reset plate projects further away from the side surface of the housing. In this position, the reset plate can be pushed to cause the cam surface to move the follower to move from left to right, from the perspective of FIG. 4 to reset the primary spring.

In an example, the piston can move along the first axis or longitudinal axis when the trigger is pressed. For example, when the trigger is pressed, the trigger tip moves away from the follower to allow the piston to advance against the rear surface of the stepped barrel. This can move the barrel the same displacement amount to displace the plunger within the ampule.

Thus, following a trigger, the piston can push against the stepped barrel to allow one of the steps to push against the plunger to then propel a quantity of fluid out the barrel. The stepped barrel can return to the pre-trigger position by utilizing a spring to push against the front surface of the barrel. Then as carrier rotate, as discussed above, the next step is placed adjacent the proximal end surface of the plunger to take up the gap or distance that the plunger is just moved in response to the immediate prior injection.

Methods of making and of using the needleless injector and components thereof discussed herein are within the scope of the present invention.

### BRIEF DESCRIPTION OF THE DRAWINGS

These and other features and advantages of the present devices, systems, and methods will become appreciated as the same becomes better understood with reference to the specification, claims and appended drawings wherein:

FIG. 1 is a cross-sectional side view of a needleless injector provided in accordance with aspects of the invention.

FIG. 2 is an end cross-sectional view of the needleless injector through the stepped barrel of the carrier.

FIG. 3 is an end cross-sectional view of the needleless injector through the gear at the stem of the carrier.

FIG. 4 is a cross-sectional side view of the needleless injector of FIG. 1 following a trigger.

FIG. 5 is an end cross-sectional view of the needleless injector through the stepped barrel of the carrier, similar to FIG. 2.

FIG. 6 is an end cross-sectional view of the needleless injector through the gear at the stem of the carrier, similar to FIG. 3.

FIG. 7 is an enlarged view through the gear of the stem of the carrier of FIG. 3 or FIG. 6.

FIG. 8 is a schematic view of a carrier and an ampule to illustrate the multi-shot concept of the present invention.

### DETAILED DESCRIPTION

The detailed description set forth below in connection with the appended drawings is intended as a description of the presently preferred embodiments of needleless injectors provided in accordance with aspects of the present devices, systems, and methods and is not intended to represent the only forms in which the present devices, systems, and methods may be constructed or utilized. The description sets forth the features and the steps for constructing and using the embodiments of the present devices, systems, and methods in connection with the illustrated embodiments. It is to be understood, however, that the same or equivalent functions and structures may be accomplished by different embodiments that are also intended to be encompassed within the spirit and scope of the present disclosure. As denoted elsewhere herein, like element numbers are intended to indicate like or similar elements or features.

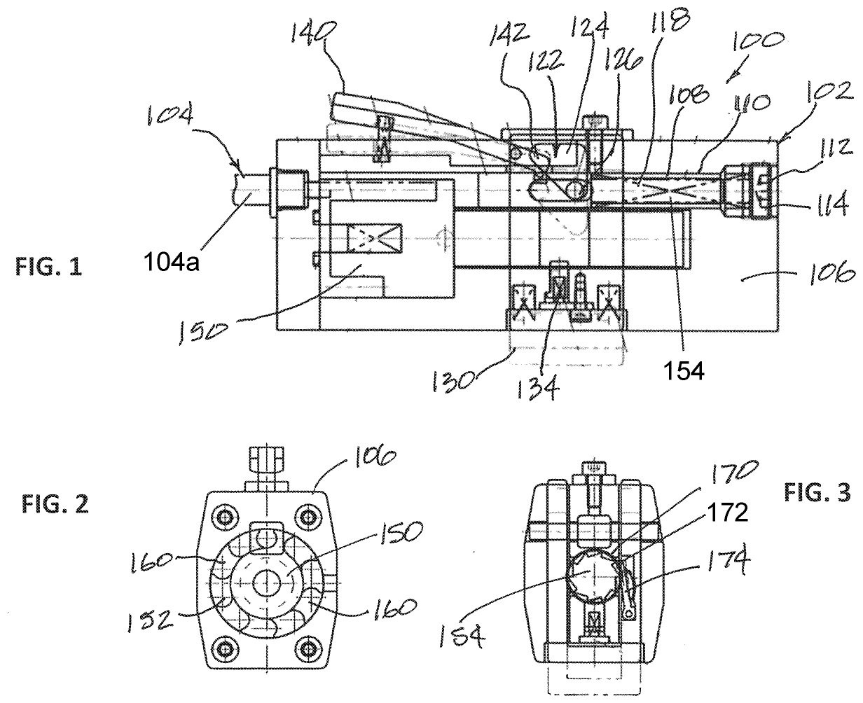

With reference now to FIG. 1, a lengthwise cross-sectional side view of a needleless injector 100 provided in accordance with aspects of the invention is shown, which has a drive end 102 and an ampule 104, which is only partially shown. The ampule 104 may embody prior art ampules, which has an ampule housing 104a with an engagement end for attaching to the drive end 102 and a remote end, which has a discharge tip. A plunger with a plunger tip is slidably disposed inside the ampule housing 104a. When the plunger is pushed or impacted by a piston of the drive end 102, the piston accelerates the plunger in the distal direction inside the ampule housing to then dispense a quantity of fluid, such as medicine or other injectable fluids, out the discharge tip. The ampule and plunger are schematically shown in FIG. 8, as further discussed below.

The drive end 102, which may also be referred to as an injector or an injection driver, is provided with a pressure or power source for driving a piston against the plunger of the ampule to discharge a quantity of fluid contained inside the ampule. The ampule 104, which can be similar to one of the ampules described in WO 2014/042930, PCT/US2013/058013, the contents of which are expressly incorporated herein by reference, can have a barrel and a movable plunger located therein. The drive end 102 of the present invention is configured for moving or displacing a piston in increments so that the piston can move against the plunger of the ampule 104 in increments to deliver fluids contained inside the barrel of the ampule in distinct dosages. Said differently, the present drive end is configured to drive a piston in multiple increments to then drive the plunger in increments to discharge fluids inside the ampule in finite quantities or dosages.

In an example, the drive end 102 comprises a housing 106 having a plurality of components contained therein. In an example, the housing 106 is sized and shaped to accommodate a helical spring 108, which can be a compression spring, located inside a spring chamber 110, which can be closed off by a set screw or end cap 112. In an example, the set crew 112 can be adjusted within a recessed chamber 114 to control the depth of how far the set screw seats within the chamber to then control the amount of compression of the spring 108, which allows some adjustability of the spring along the spring curve, and therefore the force that the spring exerts against the piston 118. In an example, the piston is position through the coil center of the plurality of coils of the spring 108. The end cap 112 can be adjusted, such as threaded, to move distally or proximally within the recessed chamber 114.

The piston 118 is aligned to a cam and follower assembly 122, which has a cam 124 and a follower 126. In the example shown, the cam 124 can have a structure with a cam surface that resembles a hypotenuse of a right triangle. The follower 126 can be a rod, shaft or pin and can be in dynamic communication with the cam surface. If the housing has a lengthwise axis, the follower 126 can be configured to traverse along the lengthwise axis while the cam is configured to displace radially of the lengthwise axis. For discussion purposes, the lengthwise axis may be called the first axis and movement radial of the lengthwise axis may be called a second axis. This arrangement allows the cam 124 to move along the second axis to cause the follower 126 to move in the first axis, and vice-versa.

Thus, the follower 126 is configured to move a lengthwise distance equal to the two ends of the slot of the cam 124, which can be called or labelled a shot displacement distance. That is, when the drive end 102 is triggered to release the spring 108, the piston 118 is configured to move a shot displacement distance to move the follower 126 the same shot displacement distance.

In an example, the follower 126 can be secured to the piston 118. For example, the follower can project through an opening on the piston. Thus, movement of the piston 118, the follower 126, and the cam 124 can be tied together or dependent on one another. In an example, there can be two similar cams 124 located on either side of the piston 118. For example, each cam 124 can embody a generally rectangular plate that extends about the width of the housing 106. Each plate can have a cut-out having a cam surface. Each plate can displace along the second axis, perpendicular to the lengthwise axis of the housing. Thus, after the piston 118 is triggered and move along the first axis, or lengthwise axis of the housing, the cams are permitted to move along the second axis. Then as the cams 124 are pushed along the second axis following the trigger, actions of the cams are used to cause the follower 126 to move along the first axis to reset the spring 108 to enable a second trigger, and so forth.

In an example, a reset plate 130 is provided to reset the cams 124. The reset plate 130 can be secured to the two cams 124 via a plurality of set screws or another fastener means. One or more compression springs 134 can be provided between the reset plate 130 and a shoulder formed inside the housing 106, which may be considered or called a return spring or return springs, depending if more than one return spring is used. The return springs 134 push the reset plate 130 outwardly, as shown by the dashed lines in FIGS. 1 and 3, to provide the user with a projected or protruded surface for resetting the primary spring 108. For example, pressing on the reset plate 130 imparts a force along the second or radial axis. This then causes the follower 126 to move from the left most position inside the cam to a right most position inside the cam or causes the follower to move along the first axis, or along the lengthwise axis. This then causes the piston to also move from left to right, when viewing FIG. 1, to reset the primary spring 108. A pivotable trigger 140 can be provided to maintain the spring 108 in the loaded or cocked position. For example, a trigger tip 142 can be provided to abut the cam or a structure connected to the cam to retain the cam until the trigger 140 is depressed or triggered.

FIG. 8 shows a schematic diagram of components, such as a carrier 150, of the needleless injector 100 of FIG. 1. With reference to FIGS. 1 and 8, the carrier 150 comprises a stepped barrel 152 and a stem 154. The carrier 150 is provided inside the housing 106 and can be used to generate discrete axial displacements of the plunger located inside the ampule. The stepped barrel 152 has a front surface 156 and a rear surface 158 defining a length therebetween. Within the barrel 152 or formed as part of the body of the barrel, steps 160 can be provided similar to steps on a winding staircase. The gaps between each step 160 of the multiple steps on the stepped barrel defines a length along the first axis. Movement of the barrel can cause the movement of the plunger 164 having a plunger tip 164a a shot displacement distance, which can be sized to deliver a desired quantity or volume of fluid out the barrel. For example, at the start of an injection cycle when the barrel 162 is filled with a fluid to be injected and the plunger 164 is at its most rearward or proximal position, the plunger can seat against the lowest, or right most, step 160a of the carrier. After the plunger 164 is advanced a shot displacement distance by the piston 118 as the spring 108 is released by the trigger 140, the plunger 164 moves deeper into the barrel by approximately the same amount within the barrel to displace a quantity of fluid out the distal end of the barrel.

When the spring is reset by pressing on the reset plate 130 discussed above, the stem 154 is rotated so that the next step 160b is rotated to be next to the proximal end of the plunger 164. The gap or distance between the two steps 160a, 160b is approximately equal to the shot displacement distance. Then after the plunger 164 is again advanced a shot displacement distance by the piston 118, the plunger 164 moves further deeper into the barrel by approximately the same amount within the barrel to displace a second quantity of fluid out the distal end of the barrel. When the drive end is again reset by pressing on the reset plate 130, the stem 154 is rotated to rotate the stepped barrel 152 to move the next step 160c adjacent the proximal end of the plunger 164, which has been displaced in the prior step. At this position, the drive end can fire again to deliver a third shot out the distal end of the barrel without a needle. The process can repeat until the barrel is spent or completely discharged of fluids contained therein. The number of shots can be designed into the barrel by providing the number of steps for generating the number of shots. As shown in FIG. 8, three shots can be generated with the three steps 160a, 160b, 160c. However, fewer or more than three steps can be incorporated.

In an example, there can be two or more steps 160 formed with the stepped barrel 152. For example, there can be four steps for performing four injections or for displacing four distinct shots or quantities of fluids out the distal end of the barrel. Said differently, there can be four steps so that a contact surface of each of the four steps can push against proximal end most of the plunger 164 to deliver four distinct fluid deposits out the distal end of the ampule 162. In other examples, there can be more than four steps, such as six steps, eight steps, or ten steps.

FIGS. 2 and 3 show end cross-sectional views of the needleless injector 100 through the stepped barrel 152 and through the stem 154.

In an example, the stem 154 comprises a gear 170 comprising a plurality of teeth 172, or multiple individual tooth 172. The gear 170 is configured to cooperate with the cams 124, or with one or more levers formed with the one or more cams 124. When the cams 124 are pushed to reset the spring 108, as discussed above, the one or more levers 176 push against the gear 170 to rotate the stem 154 to then rotate the stepped barrel so that the next increment step is rotated for pushing against the proximal end edge of the plunger 164. In an example, each reset of the spring 108 causes the one or more levers on the cam 124 to push against one tooth 172 to rotate the stem. In other words, each tooth can be pushed serially one at a time during each reset of the spring.

As discussed, while the plunger 164 moves deeper into the barrel 162 following each injection, generated by propelling the stepped barrel 152 with the spring 108 when the trigger 140 is pressed, the present needleless injector 100 can be reset to perform multiple injections with the same ampule 162 such that the first injection and the last injection among the two more injections can be performed with the same drive end 102 without having to remove the ampule 162 in between two successive injections. Further, by controlling the shot displacement distance and the spacing between two different steps 160 on the stepped barrel 152, the multiple shots can each have roughly the same quantity or volume of fluid dispensed out the discharge end of the barrel.

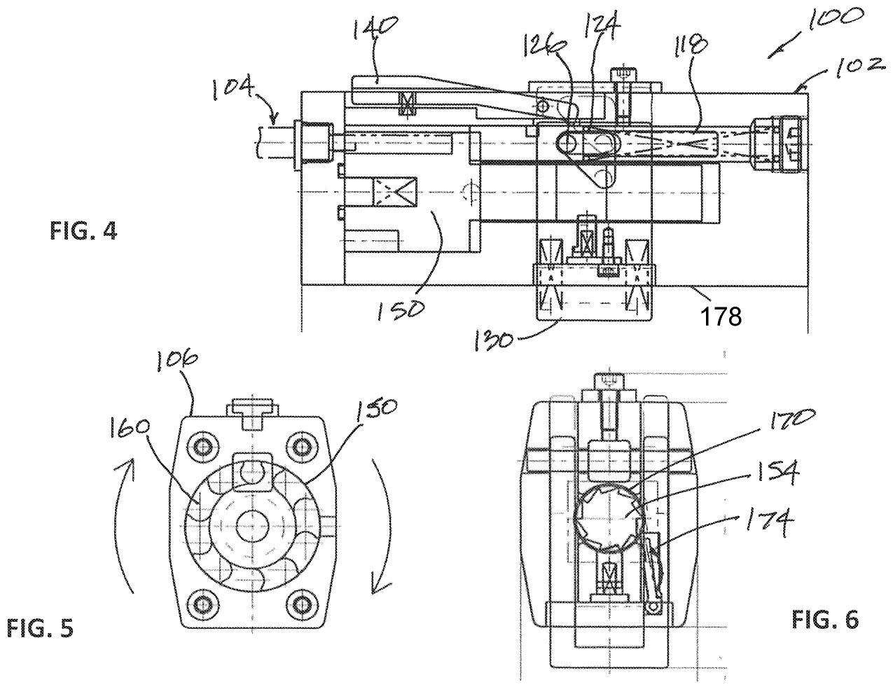

FIG. 4 is a cross-sectional side view of the needleless injector 100 following an injection. As shown, the trigger 140 is depressed and the trigger tip 142 is spaced from the cam 124, which has been displaced outwardly so that the reset plate 130 projects further away from the side surface 178 of the housing 106. In this position, the reset plate 130 can be pushed to cause the cam surface to move the follower 126 to move from left to right, from the perspective of FIG. 4 to reset the primary spring 108.

FIG. 5 shows the stepped barrel 152 of the carrier 150 rotated clockwise from the position of FIG. 2 upon resetting the cam. Similarly, FIG. 6 shows the gear 170 of the stem 154 rotated clockwise from the position of FIG. 3 upon resetting the cam.

In an example, the piston 118 can move along the first axis or longitudinal axis when the trigger 140 is pressed. For example, when the trigger is pressed, the trigger tip moves away from the follower to allow the piston to advance against the rear surface 158 (FIG. 8) of the stepped barrel. This moves the barrel the same displacement amount to displace the plunger within the ampule. Thus, following a trigger, the piston 118 pushes against the stepped barrel 152 to allow one of the steps 160 to push against the plunger 164 to then propel a quantity of fluid out the barrel 162. The stepped barrel 152 can return to the pre-trigger position by utilizing a spring to push against the front surface 156 of the barrel. Then as carrier rotate, as discussed above, the next step 160 is placed adjacent the proximal end surface of the plunger 164 to take up the gap or distance that the plunger is just moved in response to the immediate prior injection.

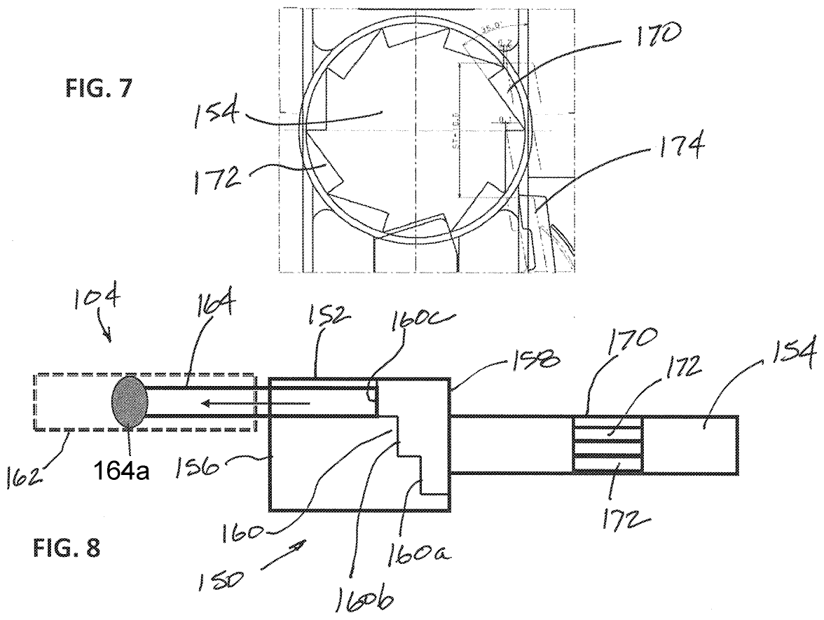

FIG. 7 is an enlarged view of the gear 170 of FIG. 3, which is similar to FIG. 5.

Methods of making and of using the needleless injector and components thereof are within the scope of the present invention.

Although limited embodiments of needleless injectors and their components have been specifically described and illustrated herein, many modifications and variations will be apparent to those skilled in the art. Accordingly, it is to be understood that the needless injectors and their components constructed according to principles of the disclosed devices, systems, and methods may be embodied other than as specifically described herein. The disclosure is also defined in the following claims

What is claimed is:

1. A needleless injector for dispensing a quantity of fluid without a needle, said needleless injector comprising:
a drive end comprising a housing,
a piston, a compression spring, and a cam and follower assembly located inside the piston; and
wherein a follower of the cam and follower assembly is arranged to displace along a first direction and a cam of the cam and follower assembly is arranged to displace along a second direction, angled to the first direction.

2. The needleless injector of claim 1, wherein the cam comprises a cam surface for moving the follower along first direction, which is parallel with a lengthwise axis of the housing.

3. The needleless injector of claim 1, further comprising a carrier comprising a stepped barrel and a stem in dynamic arrangement with the piston.

4. The needleless injector of claim 3, wherein the stepped barrel comprises a plurality of steps and wherein a trigger length between a first step and a second step is approximately equal to a trigger length between the second step and a third step.

5. The needleless injector of claim 4, wherein the piston is displaceable along a first axis an amount that is substantially equal to the trigger length between the first step and the second step.

6. The needleless injector of claim 1, further comprising an ampule attached to an opening of the housing.

7. The needleless injector of claim 3, further comprising a stem extending from the stepped barrel, said stem comprising a gear.

8. The needleless injector of claim 1, further comprising reset plate extending out a side of the housing.

9. The needleless injector of claim 8, further comprising one or more springs biased against the reset plate.

10. The needleless injector of claim 3, wherein the piston abuts against a rear surface of the stepped barrel.

11. A method of performing multiple injections using a needleless injector, said method comprising pushing a trigger to perform a first injection, pushing a cam to reset a spring, and pushing the trigger to perform a second injection.

12. The method of claim 11, further comprising rotating a carrier comprising a stepped barrel, said stepped barrier in contact with a plunger.

13. The method of claim 11, wherein the first injection and the second injection are performed with a same ampule, said ampule comprising a barrel and a plunger.

14. The method of claim 13, further comprising pushing on the cam to reset the spring, and pushing the trigger to perform a third injection with the ampule.

15. A method of making a needleless injector, said method comprising:
assembling an ampule to a housing of a drive end; wherein
said ampule comprises a plunger with a plunger tip slidably disposed in an interior of an ampule housing;
said housing comprising a piston, a compression spring, and a cam and follower located inside the piston; and
wherein a follower of the cam and follower assembly is arranged to displace along a first direction and a cam of the cam and follower assembly is arranged to displace along a second direction, angled to the first direction.
